(19) United States
(12) Patent Application Publication
(10) Pub. No.: US 2011/0059498 A1
(43) Pub. Date: Mar. 10, 2011

(54) Method and Device for Producing Electricity and Conversion Products, Such as Ethanol

(76) Inventors: Johan Pieter Marinus Sanders, Groningen (NL); Koen Peter Henri Meesters, Amersfoort (NL); Johannes Gerardus Bernardus Van Der Broek, Tynaarlo (NL)

(21) Appl. No.: 12/919,342

(22) PCT Filed: Mar. 6, 2009
(86) PCT No.: PCT/NL2009/000056

§ 371 Date: Nov. 23, 2010

(30) Foreign Application Priority Data
Mar. 6, 2008 (EP) EPO8075175

(51) Int. Cl. C12P 7/06; C12M 1/00; C12P 5/02

(52) U.S. Cl. 435/161; 435/289.1; 435/167

(57) ABSTRACT

The invention relates to a method for producing electricity and conversion products such as ethanol, comprising the steps of: i) separating a starch source into a starch-rich fraction and a residual fraction; ii) heating the starch-rich fraction for the purpose of gelling the starch; iii) releasing the gelled starch from the starch-rich fraction; iva) converting the gelled starch enzymatically into sugars; ivb) converting the sugars fermentatively into the conversion products; v) further processing the conversion products from the conversion medium; vi) generating biogas from residual fraction; vii) generating electricity and heat from biogas and/or residual fraction via cogeneration of heat and electricity; and viii) using the generated heat in one or more steps i) to vi).

The present invention relates to a method and a device for producing electricity and ethanol.

There is at the moment a growing need for alternatives to fossil fuels, particularly for generating electricity and biofuel. One possibility is the use of renewable energy sources. In order to allow the production of electricity and biofuel from renewable energy sources in such a way to take place on the soundest possible economic basis, it is important to take the fullest advantage of these environmentally-friendly energy sources and to produce, in addition to electricity, other valuable products, in this case ethanol. In order to take full advantage of these renewable energy sources it is important to make use of cogeneration of heat and electricity, whereby electricity or mechanical power is produced simultaneously with useful thermal energy. This greatly improves the utilization of the renewable energy source.

The present invention is based on the insight that, by optimal utilization of a starch source in combination with cogeneration of heat and electricity, a method and device for producing electricity and conversion products is obtained which meets the above stated objectives. The optimal utilization of the starch source consists on the one hand of an adequate separation of the starch source into a starch-rich fraction and a residual fraction. The starch-rich fraction is used to prepare microbiological conversion products, such as ethanol, butanol, citric acid, lactic acid and derived products such as calcium lactate. The residual fraction is used for direct generation of electricity and heat, as well as indirectly via biogas. The heat generated during the cogeneration of heat and electricity is used in the processing and converting of the starch source into starch and conversion products, of the residual fraction for the purpose of generating biogas, and of diverse recycling flows.

The present invention therefore provides a method for producing electricity and conversion products such as ethanol, comprising the steps of:

i) separating a starch source into a starch-rich fraction and a residual fraction;

ii) heating the starch-rich fraction for the purpose of gelling the starch;

iii) releasing the gelled starch from the starch-rich fraction;

iva) converting the gelled starch enzymatically into sugars;

ivb) converting the sugars fermentatively into the conversion products;

v) further processing the conversion products from the conversion medium;

vi) generating biogas from residual fraction;

vii) generating electricity and heat from biogas and/or residual fraction via cogeneration of heat and electricity; and

viii) using the generated heat in one or more steps i) to vi).

In a preferred embodiment the starch-rich fraction comprises starch-rich plant organs, preferably kernels, seeds or grains, but also tubers or roots. The starch source from which the starch-rich organs are obtained is chosen here such that a starch-rich fraction from the starch source, for instance a starch storage organ of the plant, such as a kernel, a tuber, a root and parts thereof, can be separated from a residual fraction in relatively simple manner and preferably using existing techniques. This obtained starch-rich fraction, preferably a kernel fraction, also has the advantage that because of its natural origin the starch is enclosed in a biological covering such as a starch storage organ. It is particularly in this biological covering that the starch is gelled through heating and thereby becomes more readily available for enzymatic conversion than crude starch. It will be apparent that in the heating for the purpose of gelling the starch use is made of thermal heat from the combined heat and power system. Available heat with a relatively low economic value is thus usefully employed.

The great advantage of gelling the starch in its natural covering is the avoidance of a buildup of viscosity. The gelling in the starch-rich fraction and, following release from the starch-rich fraction, the subsequent enzymatic conversion of starch can hereby take place in relatively simple equipment, such as a tank. Nor does water have to be added to control the viscosity. An ethanol flow of 25% by volume taken over the whole process can hereby be made from for instance maize kernels with 50% dry matter (DM) without meanwhile adding water.

At higher ethanol concentrations the released waste water flow is smaller than in other processes where water is added. This has the advantage that less energy and investment is necessary for the purpose of converting the ethanol and other products.

A preferred embodiment of the invention comprises of bringing the starch-rich fraction, in particular the starch-rich plant organs, into contact with an aqueous medium prior to gelling of starch. Such a treatment of the starch-rich organs in water, or any other suitable aqueous medium, results in maceration, or absorption of water and softening or hydration thereof, this resulting in more efficient gelling of the starch and even more efficient progress of following steps in the process according to the invention, such as production of fermentable sugars, fermentation, ethanol production or production of biogas. The time gain occurring when the plant organs are softened prior to gelling can be significant. Maceration of the starch-rich plant organs preferably takes place for 12 to 48 hours. Maceration of such organs can take place in water at a temperature suitable for the purpose. A suitable temperature for this purpose is a temperature of between 4° C. and room temperature (18° C.-23° C.), wherein room temperature is preferred.

The advantageous effects of maceration and gelling of the starch in starch-rich plant organs can be further enhanced by freezing and/or grinding macerated and gelled starch-rich plant organs.

After cooling of the gelled starch in its covering, the gelled starch is removed from its covering using existing simple techniques such as mechanical pressing using a screw press or a crusher.

The enzymatic conversion of the gelled starch into sugars then takes place. Classic enzymes or enzyme systems can be applied for this enzymatic conversion. These enzymes comprise amylase as well as amyloglucosidases. Combined enzyme systems can also be applied. It will be apparent that the thermal heat possibly required in this enzymatic conversion will be supplied from the combined heat and power system.

It is also possible to add to the product fermentation available residual flows comprising sugars, such as beetroot juice, grass juice, potato juice, stale bread (steamed), potato peelings, calf, pig, cattle, poultry manure. The desired conversion products are formed in this product fermentation.

The conversion products, such as ethanol, formed by means of the fermentation are then removed from the conversion medium and discharged as valuable product.

The remaining medium comprising starch, yeasts, enzymes (possibly bound to starch, such as amylase) is fed back to the product fermentation. A final residual flow is discharged to the biogas fermentation.

The residual fraction resulting from separation of the starch-rich fraction from the starch source can be applied to generate electricity by direct combustion or indirectly by subjecting the residual fraction wholly or partially to a biogas production (biogas fermentation), and by subsequently using the biogas to generate electricity or mechanical power.

Suitable as starch source which meets the requirement of separating a starch-rich fraction wherein the starch is present in its biological covering, such as a kernel, tuber or root, are maize, wheat, sorghum, potato, triticale, cassaya, rice, batata. The starch source is in fact the plant, such as the maize plant and the cereal plant, from which the maize kernel, cereal grain or other grain, tuber, root is separated from the remaining plant material forming the residual fraction. This means that from the starch-rich fraction a high-value end product (ethanol) can be obtained which is competitive at the price level of oil and is of a greater economic value than the energy and/or heat to be generated with this starch-rich fraction (which should be able to compete with coal). The less economically valuable residual fraction can however be used to generate biogas which competes at macroeconomic level with coal or natural gas. The biogas or the residual fraction can be burned in a combustion plant for generating energy and heat. The advantage of using the maize plant is the relatively high starch yield (8 tonnes per 18 tonnes of dry matter per hectare). The maize plant is thus a preferred starch source for the method according to the present invention.

Because the starch source is generally not available all year round while the production of conversion products, in particular ethanol, and electricity/heat production are desired throughout the year, it is recommended that the starch-rich fraction is stored, optionally after a preservative treatment. Because the starch-rich fraction has been subjected to a treatment and can be stored for a longer period of time, this starch-rich fraction is available throughout the year for the purpose of ethanol production. Such a preservative treatment can for instance be performed by means of a treatment with propionic acid or a treatment with CO₂, wherein the CO₂ can be recovered from the fermentation reaction as described herein, after which the starch-rich fraction can be stored, for instance by ensilaging. Another form of pretreatment is to increase the dry matter content, thereby improving the storing quality. The dry matter content can for instance be increased by drying, while another option is to leave the crop longer in the field.

The treatment with propionic acid can be performed with an undiluted or diluted propionic acid solution, wherein it is recommended to use a twice diluted solution of a concentrated propionic acid solution of 95% (w/v). Propionic acid solutions diluted four times, eight times or sixteen times are also suitable for the invention.

An advantage of using a twice diluted propionic acid solution is that the starch-rich plant organs can remain stored for a period of at least eight months without adverse or undesirable effects occurring in the method according to the invention. The treatment with the diluted propionic acid solutions provides protection against the growth of undesirable micro-organisms in the stored starch-rich plant organs, without fermentation or other subsequent processes being adversely affected.

Heating of the kernel fraction takes place in relatively simple containers. Heat from the combined heat and power system is transferred via a heat exchanger to a liquid. This heated liquid is brought in the container into direct contact with the starch-rich fraction. In this way the starch-rich fraction can be heated and gelled in relatively simple containers. Because the gelling of the starch takes place in the natural covering of the starch source, no buildup of viscosity occurs in the container. Because no viscosity buildup occurs, no water need be added to reduce viscosity buildup in the gelled material. After sufficient gelling of in its natural covering, for instance a kernel, the starch is cooled.

Since no water need be added, more concentrated operation is possible. Yeast, such as Saccharomyces cerevisiae, and/or other ferments and/or other fermenting agents and/or enzymes can hereby be reused a number of times. The separation of the yeasts (and/or fermenting agents), enzymes and starch can take place using for instance a microsieve (1-10 μm), a centrifuge or a separator or decanter.

The heat generated during this cooling can be further applied in the method according to the invention. The cooled starch is then pressed out of its natural covering, for instance a kernel, for instance with a screw press or crusher.

Heating of the starch in its biological covering (kernel) preferably takes place at a relatively high temperature (high killing effect), whereby the formed gelled starch is in fact substantially aseptic or sterile. This high-killing (preferably heat) treatment whereby the number of micro-organisms present is greatly reduced) is essentially not disadvantageous for the subsequent microbiological and/or enzymatic conversions, and is recommended because use is subsequently made in the enzymatic conversion of sterile gelled starch, whereby it is possible to work with relatively low concentrations of enzymes and yeasts without a great risk of infections. This avoids a part of the starch being converted during fermentation not into desired conversion products but into undesired products.

The enzymatic conversion of gelled starch into sugars can be performed by means of fermentation wherein use is made of a classic amylase, such as BAN®, a classic thermostable amylase such as Maxamyl®, or an enzyme which can be readily applied under the same conditions as yeast fermentations (temperature between 30 and 40° C. and pH between 3 and 6) such as StarGen. The enzymatic conversion can take place relatively quickly by making use of an enzyme system comprising at least one amylase and at least one amyloglucosidase. This takes place more rapidly particularly because the starch is already gelled. The conversions can be performed in different tanks or in a shared tank.

Although already stated above, it will be apparent that thermal heat possibly required during the enzymatic reaction will come from the combined heat and power system.

Following the conversion of the gelled starch into conversion products such as ethanol, the conversion products are then further processed from the conversion medium. This further processing can take place using distillation, stripping or vacuum-stripping and pervaporation and/or particularly separating techniques which require heat for the purpose of separation. The heat is supplied by the combined heat and power system. If the method is performed on relatively small scale, use can be made of another processing technique for processing ethanol or other conversion products such as butanol. This processing technique comprises on the one hand of gas stripping followed by ethanol condensation. The gas stripping of ethanol or butanol takes place by blowing a gas into the conversion medium. Owing to its volatility, ethanol will be entrained with the gas flow and leave the conversion medium. It will be apparent that, depending on the operating pressure, temperature and the like as well as the composition of the conversion medium, ethanol can be removed to a determined extent via gas stripping. Important here are the liquid vapour equilibrium of water and the associated ethanol volatility. It will be apparent to the skilled person which process conditions must be applied in order to ensure optimum gas stripping of ethanol and other conversion products such as butanol from the conversion medium. The gas stripping can be performed with any type of inert gas (the volatility of ethanol is hardly dependent on other gases in gas phase). Recommended is CO₂ stripping, and particularly CO₂ from the fermentation of sugars to ethanol and CO₂. An additional advantage is that, after CO₂ stripping, the remaining CO₂ can be used for a further application in horticulture, and can therefore be recovered and reused for fertilizing purposes in horticultural glasshouses.

In the case of vacuum stripping the volatile conversion products (for instance ethanol) and water are separated under reduced pressure and separated from each other in a gas stripping column. After compression the vapour is cooled and condensed.

For continuous electricity and ethanol production it is also desirable that an adequate quantity of residual fraction is continually available. It is therefore recommended that the residual fraction, after harvesting and separation, is stored for instance by ensilaging. Less biomass is available for the generation of biogas as a result of the premature separation of the starch-rich fraction from the starch source. By way of compensating for the removal of easily fermentable components from the raw material for the digester, it is recommended to treat the residual fraction, whereby this residual fraction is more readily available for generating biogas. Sufficient biogas is in this way still available for existing plants for cogeneration of heat and electricity at which the ethanol production unit can be placed. A pretreatment can in particular take place, thereby resulting in a better utilization of the cellulose and lignocellulose present in the residual fraction. It is of course the case in newly constructed processing plants that the highest possible yield of all products is desirable. Particularly envisaged here is a pretreatment as described by Maas R. H. W., Thesis: Microbial conversion of lignocellulose-derived carbohydrates into bioethanol and lactic acid. Chapter 2: Mild-temperature alkaline pretreatment of wheat straw to enhance hydrolysis and fermentation, 2008, by Klaasse Bos G. J., Thermo-chemical pretreatment of lignocellulosic biomass to enhance anaerobic biodegradability, 2007, and by Klaasse Bos G. J., Optimization of biogas recovery from organic residual flows, 2007. A pretreatment using calcium hydroxide 10%, on dry matter basis, heating to 70° C.-100° C. is recommended since the efficiency of obtaining biogas from the residual fraction is thereby improved.

The digestate from the biogas fermentation can be separated into a thin and a thick fraction. The thick fraction from the biogas fermentation still comprises significant quantities of undigested cellulose and lignocellulose. By first heating a large part of the dissolved CO₂ will evaporate. The quantity of Ca(OH)₂ required in the following treatment to reach a sufficiently high pH will hereby decrease. When Ca(OH)₂ is added, ammonia will escape. The advantage hereof is that the N-content of the digestate hereby decreases. More digestate can hereby be spread on the land without exceeding environmental standards. The ammonia can be captured using an aqueous solution of acid, for instance sulphuric acid. The CO₂ that has escaped during heating and the ammonia that has escaped during the addition of Ca(OH)₂ can also be brought into contact with each other in a moist environment. Ammonium carbonate or ammonium bicarbonate can then result. In this way the ammonia can be concentrated in a small volume without making use of an acidic washing liquid. If insufficient CO₂ escapes during heating, CO₂ from the fermentation can also be applied. Feedback of the treated digestate to the biogas fermentation provides additional raw material for the biogas fermentation.

As a hygienic measure the product fermentation unit 29 can be cleaned with an alkaline agent. Such an agent is preferably Ca(OH)₂, although other suitable agents such as NaOH, KOH, Ca(O) or mixtures thereof can be used. After washing of unit 29 with such an agent, this agent can then be used for the treatment according to the invention of the thick fraction of the digestate with an alkaline agent. Use of such an alkaline agent for the purpose of cleaning the unit 29 and for the digestate treatment according to the invention results in a significant cost reduction.

As an alternative to the treatment of the thick fraction of a digestate with an alkaline agent according to the invention described herein, such a treatment can also be applied in other methods or installations for fermenting biomass.

Digestate comprises a residual fraction which is substantially not fermentable or poorly fermentable and which remains behind after fermenting of biomass. As stated, a digestate can be separated into a dry, or thick, fraction and a wet, or thin, fraction. The separation of thick and thin fractions preferably takes place with a simple rotary sieve. The thin fraction is very rich in water and comprises a low dry matter content and water-soluble minerals. The thick fraction comprises large quantities of non-fermentable or non-convertible complex polysaccharides. Such complex polysaccharides are substantially cellulose or hemicellulose (lignocellulose). Usual methods for effective further fermentation of the thick fraction of a digestate are not available. At the moment the thick fraction is used for instance as fertilizer. The object of this aspect of the intention is to provide an effective and efficient treatment of digestate. This objective is achieved by providing a method and installation for producing biogas from a thick fraction of a digestate, comprising of heating the thick fraction of a digestate, allowing CO₂ to escape from the thick fraction, adding an alkaline agent to the heated thick fraction, treating the heated thick fraction with the alkaline agent and introducing the treated thick fraction into a digester for further fermentation of the treated thick fraction.

The method according to the invention results in an increased yield of biogas, such as methane, from the thick fraction of a digestate, and therefore also from the starting material used for the purpose of fermenting biomass. The treatment of a thick fraction according to the invention has the result that sugars from the cellulose and/or hemicellulose previously not available for fermentation now do become available for fermentation thereof. Such fermentation can result in production of biogases, such as preferably methane.

Heating the thick fraction of a digestate has the result that a large quantity of CO₂ present in the thick fraction is released and escapes from the thick fraction. Because the quantity of CO₂ in the thick fraction is lower, less alkaline agent is required to increase the pH of the thick fraction to a pH suitable for causing release of previously unavailable sugars for the purpose of fermentation thereof. The temperature treatment further ensures that significant quantities of ammonia escape from the thick fraction.

Due to the treatment of a thick fraction of a digestate using an alkaline agent after heating, previously unavailable sugars are released from the thick fraction. By introducing the treated thick fraction into a digester for further fermentation of the treated thick fraction the method according to the invention provides an improved and more efficient method of producing biogas from a thick fraction of a digestate, and an increased yield of biogases such as methane or ammonia.

The digestate which can be used as starting material for the method according to the invention can come from substantially two different fermentation processes. The thick fraction of a digestate can come from a digester for biogases and from a disgester for ethanol production, as well as a mixture hereof. The biomass used as starting material for fermentation to biogases can be any material originating from plants, such as pulp, stems, leaves, fruit, kitchen and garden waste, tubers, material comprising cellulose and/or hemicellulose. Biomass used as starting material for fermentation to ethanol comprises substantially starch-rich plant organs such as seeds, kernels, tubers. It is also possible to apply the method according to the invention on a mixture of thick fractions remaining after fermentation to ethanol and/or fermentation to biogases.

In a preferred embodiment heating of the thick fraction takes place at a temperature between 70° C. and 100° C., preferably at a temperature between 80° C. and 90° C., more preferably at 85° C. When the thick fraction of the digestate is heated in accordance with the recommended temperatures, the intended degassing occurs within a short period of time. The method according to the invention hereby continues to operate in efficient manner.

In another preferred embodiment the treatment of the thick fraction with the alkaline agent takes place for 6 to 48 hours, preferably for 6 to 24 hours, most preferably for 6 to 12 hours. Such treatment times are suitable for causing release from the thick fraction to be treated of the sugars that are non-fermentable or only fermentable with difficulty. A period of between 6 and 12 hours is recommended because an effective and efficient course of the process according to the invention is hereby made possible. Longer treatment times are possible here but do not provide, or provide to lesser extent, the time gain provided by shorter treatment times.

In an embodiment of the invention the alkaline agent is chosen from the group consisting of KOH, NaOH, Ca(O) or preferably Ca(OH)₂. Such alkaline agents can be acquired cost-effectively. Such agents can also be used to clean a fermentation unit, such as unit 29 according to the invention. This results in less need for the use of alkaline agents according to the invention. This enables a cost-effective method according to the invention.

In another preferred embodiment the pH during treatment of the thick fraction is between pH 9 and 12, preferably between pH 9.5 and pH 10.5. At such a degree of acidity there occurs a rapid and appropriate conversion of the unavailable cellulose and lignocellulose into sugars for fermenting, wherein the fermentation proceeds as according to, the invention described herein.

In order to reach such a pH, in a preferred embodiment the alkaline agent is added in a concentration of between 25 and 300 g, preferably between 25 and 150 g, most preferably between 25 and 100 g of alkaline agent per kilogram of dry matter of the thick fraction. Such quantities allow of a cost-effective application of the method according to the invention. This is particularly the case when the alkaline agents are at least partially recovered from the thick fraction.

In yet another preferred embodiment of the invention nitrogen is removed from the thick fraction by means of a gas washer. It hereby becomes possible according to the invention to obtain valuable minerals from the gas flow. By preferably recovering ammonia, nitrogen is collected in the form of salt, such as in the form of ammonium sulphate, ammonium nitrate or another salt. This more efficient use of biomass also creates less digestate per tonne of starting material used for the method according to the invention.

A yeast residual flow from the product fermentation can otherwise be supplied to, and applied in, the biogas fermentation.

Another aspect of the present invention relates to a device for performing the above described method for producing electricity and ethanol. This device comprises

a unit for separating a starch source into a starch-rich fraction and a residual fraction;

a unit for heating the starch-rich fraction for the purpose of gelling the starch;

a unit for releasing gelled starch;

at least one unit for enzymatic conversion of gelled starch into sugars and for the fermentative conversion of sugars into conversion products;

a unit for producing biogas from residual fraction;

an associated combined heat and power unit for combined production of electrical or mechanical energy and heat from the residual fraction and/or biogas;

a unit for processing the conversion products; and

means for supplying heat from the combined heat and power unit to at least one of the other units of the device.

It will be apparent that using the device according to the invention, with relatively simple equipment and making use of standard techniques and cogeneration of heat and electricity, electricity and conversion products can be generated in relatively simple and economically sound manner on a small scale (up to for instance 1 MWₑ) from a specific starch source which is only available for part of the year. The starch source is made available throughout the year due to specific operations according to the invention.

The method and device according to the invention are also suitable for use on relatively small scale. It is hereby also possible to apply remaining fibre and minerals in arable farming because of relatively low transport costs. The method and device according to the invention are therefore suitable for relatively rapid implementation of the method and device in existing infrastructure.

Mentioned and other features of the method and device according to the invention will be further elucidated hereinbelow on the basis of an exemplary embodiment, which is given only by way of example without the invention being deemed limited thereto. Reference is made here to the accompanying drawings, in which:

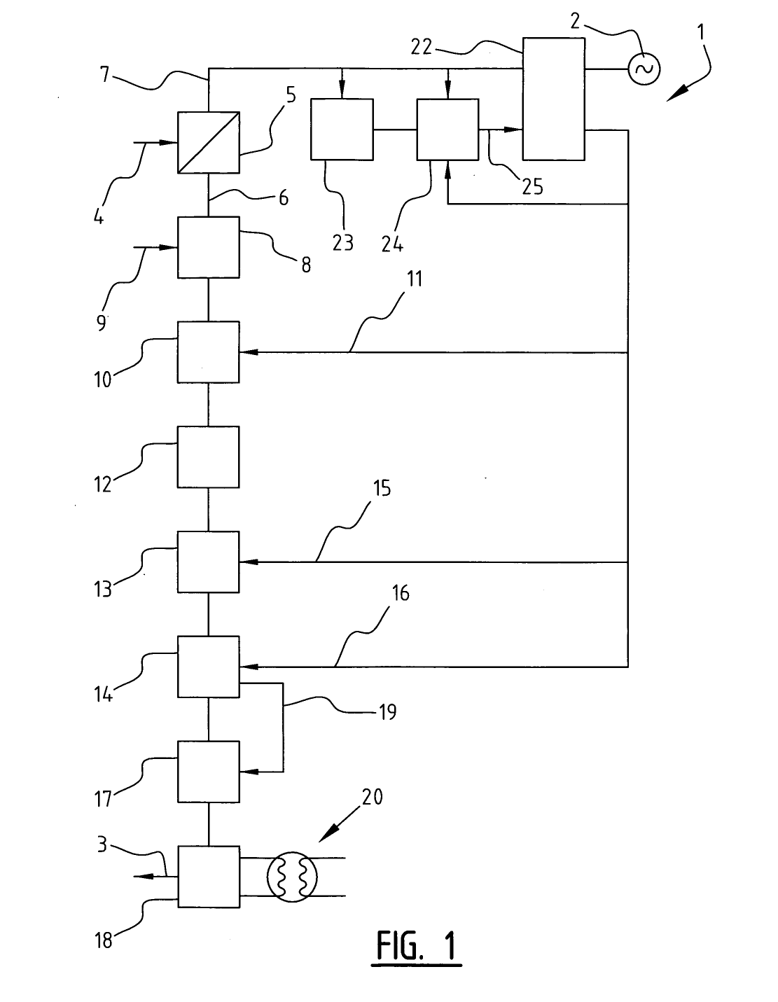

FIG. 1 shows a flow diagram of a method and device according to the invention;

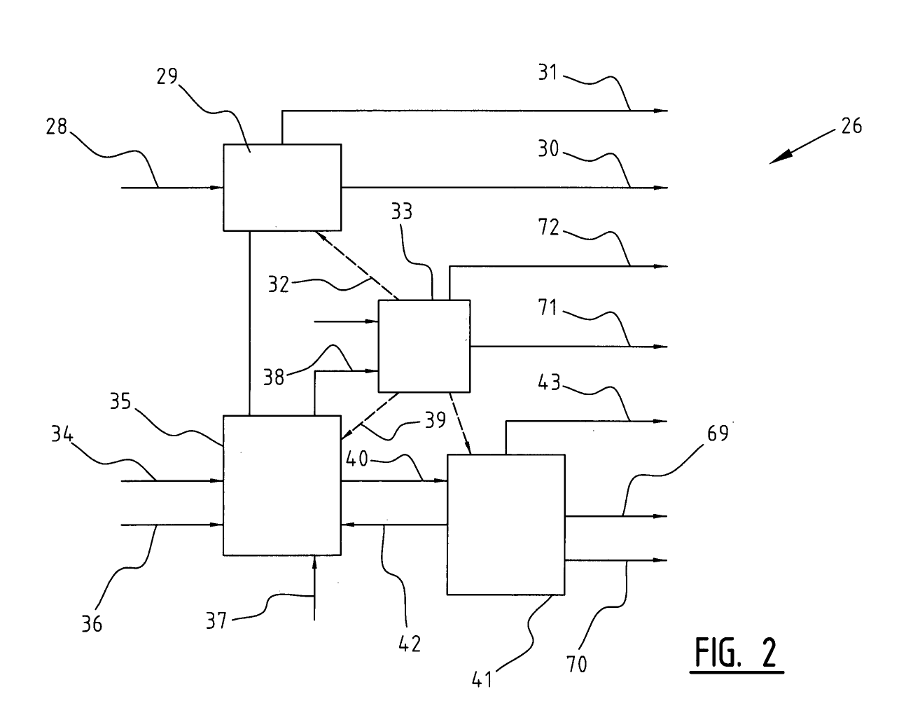

FIG. 2 shows an outline of an exemplary embodiment of the method according to the invention, wherein dashed line arrows show mass flows and dotted line arrows show heat flows;

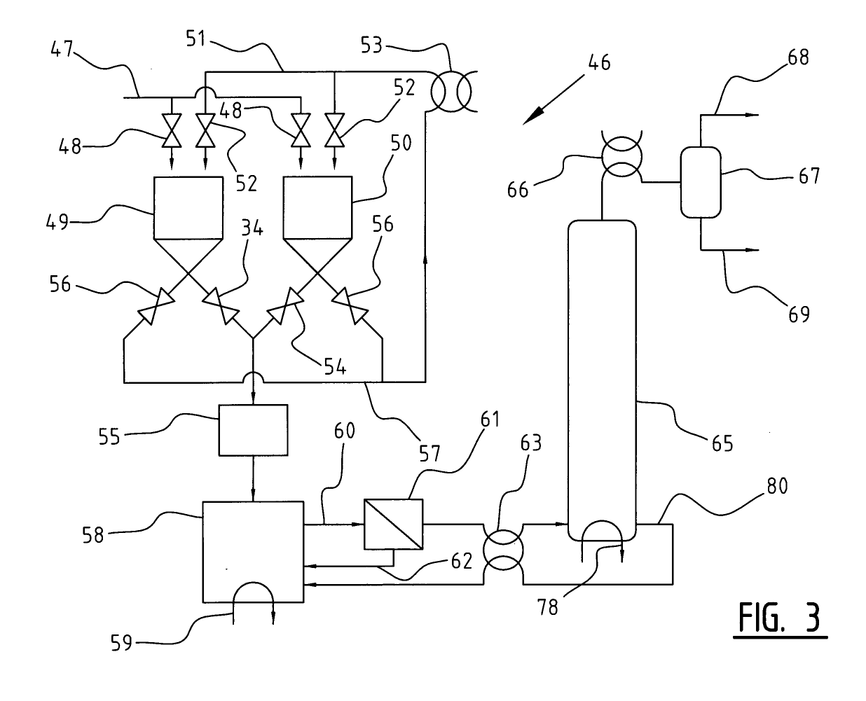

FIG. 3 shows a flow diagram of the preprocessing of the starch-rich fraction and the subsequent ethanol fermentation and distillation;

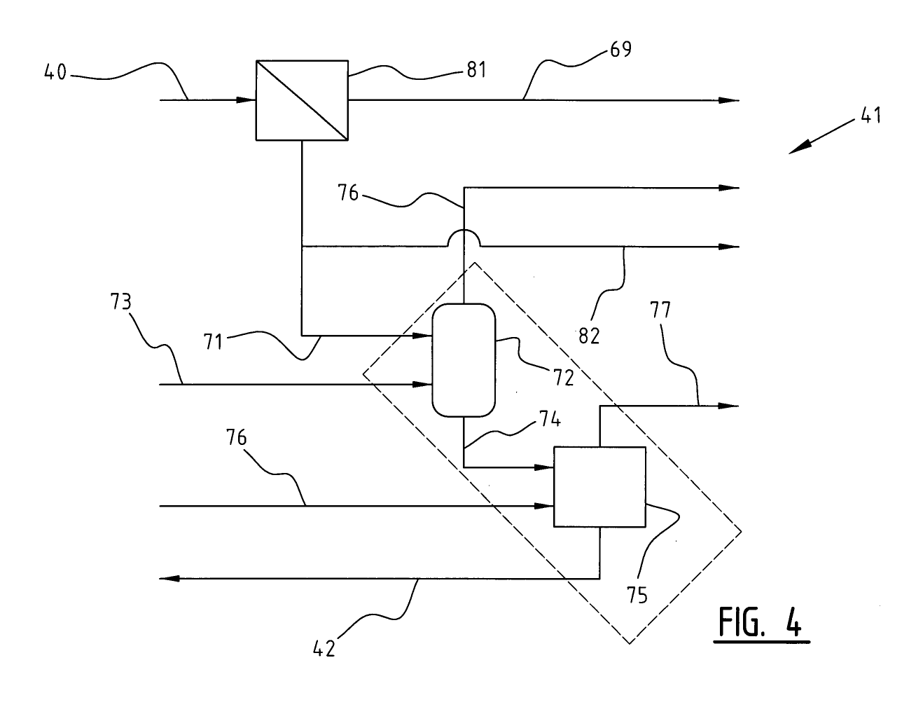

FIG. 4 shows a flow diagram of the processing of digestate originating from the biogas fermentation;

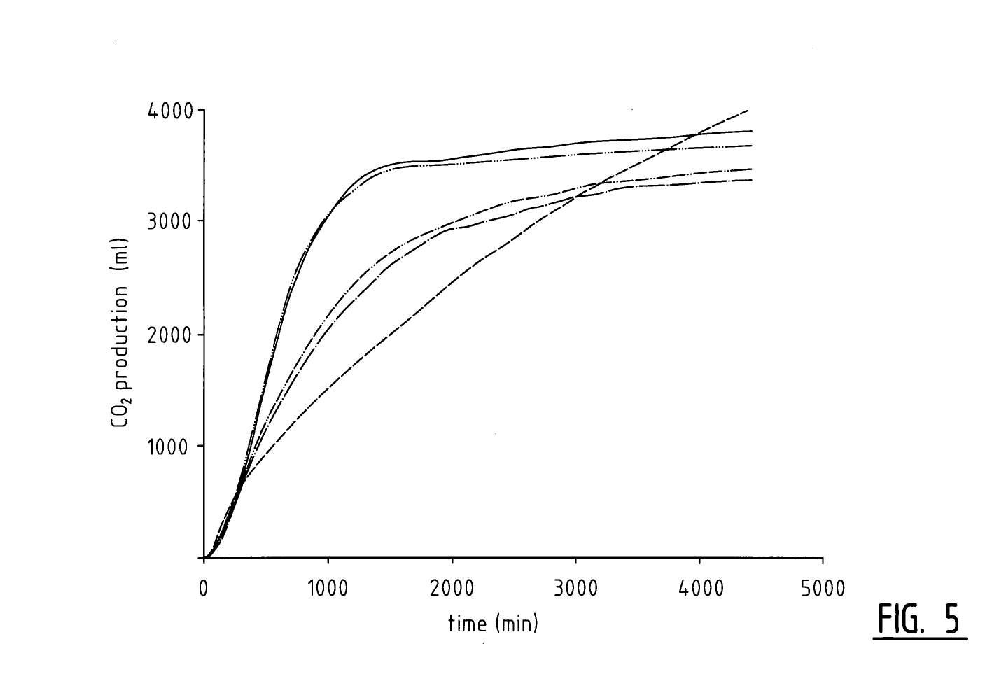

FIG. 5 shows a comparison for the CO₂ (ethanol) production for glucose (______ and - . . . -), maize starch (- . . - and - . -) and released gelled starch ( - - - ) prepared according to the invention;

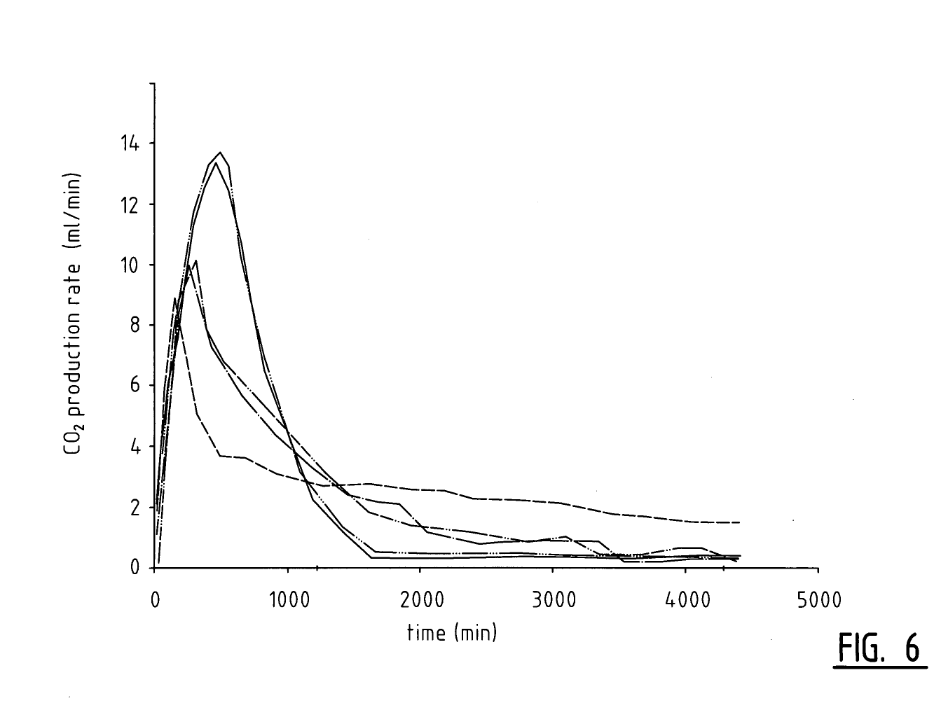

FIG. 6 shows the CO₂(ethanol) production speed;

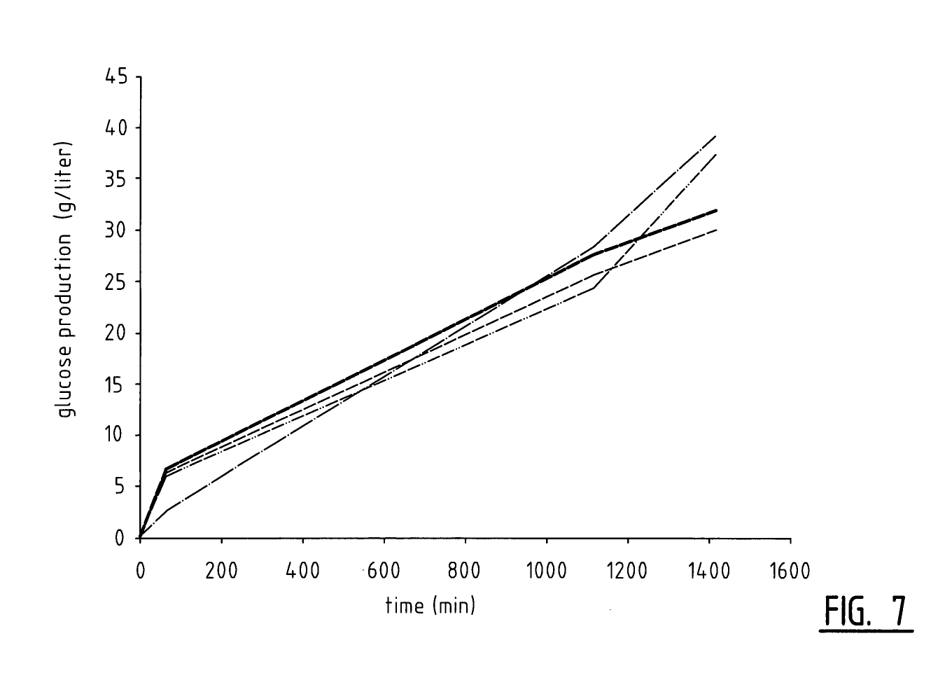

FIG. 7 shows the increase in the glucose concentration in the presence of enzymes and in the absence of yeast;

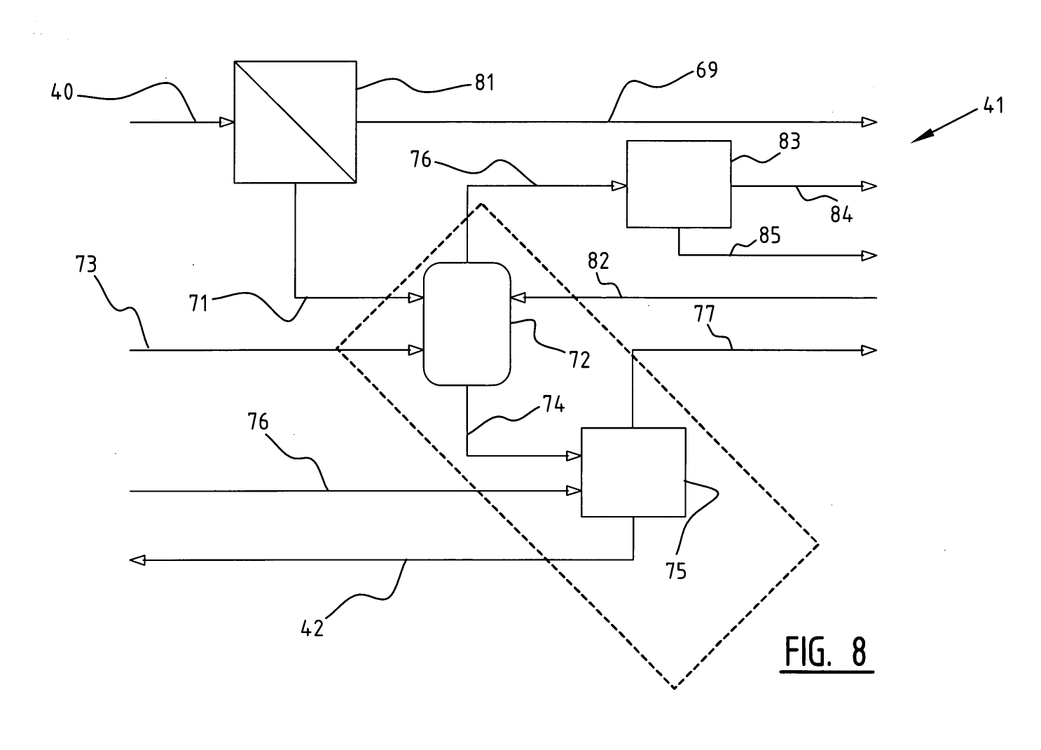

FIG. 8 shows a flow diagram of the processing of digestate originating from an alternative embodiment of the biogas fermentation.

FIG. 1 shows schematically a device 1 according to the invention for producing electricity 2 and ethanol 3.

A starch source is supplied to the device via arrow 4. This starch source can consist of maize plants, cereal plants such as wheat plants, and the like. Via arrow 4 the starch source, in this case maize plants, enters a unit 5 for separating the starch source into a starch-rich fraction 6 and a residual fraction 7. Separation between maize kernels and maize plant parts can take place in separating unit 5 using for instance a separating technique as applied in the case of cattle feed.

The maize kernels are then supplied to a storing unit 8 in which the maize kernels can be stored as required for a longer period of time before being used for further processing. A preservative can be added to storing unit 8 via arrow 9. In this case propionic acid can for instance be added. The preservative is in fact intended only to protect the maize kernels from undesirable premature degradation, while the preservative does not cause any adverse effects, particularly in the following enzymatic and biological conversion.

After storage in storage unit 8 the starch kernels are added to a heating unit 10, which in fact consists of a container provided with a heat exchanger to which heat is supplied via arrow 11. In heating unit 10 the maize kernels are heated using hot water to for instance a temperature of 60° C. to 120° C. This temperature is chosen such that the starch gels in the maize kernels and does not come out of the maize kernels. A viscosity increase in heating unit 10 is thus avoided, and the heating unit can therefore be of relatively simple construction. The heating preferably takes place such that, due to a high killing effect, the gelled starch becomes substantially sterile (has a relatively low germ count).

After sufficient heating for the purpose of gelling the starch to form gelled sterile starch, wherein the gelling can take place for a time suitable for the purpose of between 5 and 60 minutes, though preferably between 10 and 30 minutes, wherein gelling takes place at a temperature suitable for the purpose, such as between 60° C. and 120° C., preferably between 80° C. and 100° C., the starch is then supplied to a unit 12 in which the gelled starch is released from the maize kernel using mechanical means. Unit 12 can for instance consist of a gear pump, optionally with cutter, a screw press or other type of press or cutter. It is important here that during the release of the gelled sterile starch the starch remains as sterile as possible and can be supplied directly to the subsequent units for enzymatic conversion of the gelled starch removed from the starch kernel. The natural covering is here reduced in size such that the gelled starch is readily accessible to the enzymes and yeasts of the fermentation process.

The enzymatic conversion of the sterile, gelled starch takes place in unit 13. Unit 13 comprises of an enzymatic conversion of the sterile starch into glucose using digestive enzymes such as amylase and glucamylase. The formed glucose is then fed to unit 14, in which the glucose is converted fermentatively into ethanol by yeast fermentation.

As indicated in FIG. 1, heat is supplied (if necessary) to units 13 and 14 via arrow 15 and arrow 16 respectively in order to realize optimum conditions for converting starch via glucose to ethanol.

The formed ethanol is then fed to a unit 17 for processing of the produced ethanol. Unit 17 comprises a CO₂ stripper with which ethanol present in the conversion medium is volatilized by stripping with CO₂. This CO₂ flow containing ethanol is fed to a unit 18. The CO₂ required during the CO₂ stripping comes from fermentation unit 14 in which the glucose is converted into ethanol and, among other products, CO₂. This CO₂ is fed via a bypass conduit to the gas stripper of processing unit 17. The remaining conversion medium can be reused (because the supplied gelled starch was sterile) or used to generate biogas or be used directly to generate energy. Unit 18 comprises a condensation unit for the purpose of condensing the ethanol from the CO₂ gas flow which can be fed back to the gas stripper. The condensed ethanol has a concentration of generally 10 to 60% by volume, for instance 15 to 35% by volume, such as 17% by volume. A concentration can generally be obtained such that a good storage quality is obtained (from about 15% by volume to 99% by volume).

This ethanol can serve as raw material for an ethanol plant for processing of ethanol to a higher concentration and further specification. The supplied ethanol fraction can comprise traces of organic compounds, such as organic acids which need not be removed at this moment in device 1 according to the invention.

FIG. 1 further shows that residual fraction 7 can be added directly to a burner unit 22 for the purpose of generating electricity 2. It is however recommended as shown in FIG. 1 to supply the residual fraction to a storage unit 23, from which the residual fraction can be added directly as required to burner unit 22 of the combined heat and power system. In order to take full advantage of the residual fraction it is however recommended to supply the residual fraction to a pretreatment unit 23 in which the residual fraction is pretreated for the purpose of a better utilization of the lignocellulose present in the residual fraction in a unit 24 connected thereto for generating biogas. It will be apparent that residual fraction which has been pretreated or which is not pretreated can be supplied to the biogas unit. The generated biogas can be fed directly as according to arrow 25 to the burner of the combined heat and power system 22. Electricity and mechanical/thermal energy are generated in combined manner in the combined heat and power system. As shown in FIG. 1, the thermal energy can be used in the diverse units, particularly also in biogas unit 24. Although not shown in FIG. 1, it will be apparent that other residual fractions from the method and device according to the invention can also be added to the biogas unit, as well as other raw material flows such as manure and other agricultural residual products for the purpose of further optimum utilization in biogas unit 24.

Not shown is that a residual fraction from the product fermentation in unit 14 can be fed to the biogas fermentation in unit 24.

As shown clearly in FIG. 1, it is possible to generate energy cost-effectively on relatively small scale and with a relatively simple unit and to employ the heat formed therein, via cogeneration of heat and electricity, in the production of biofuel, the preprocessing of the residual fraction, the treatment of digestate and the production of biogas. A maximum use of residual heat is also guaranteed.

FIG. 2 shows an outline of a method and device 26 according to the invention. The starch-rich fraction (maize kernels) is supplied via conduit 28 to the product fermentation in unit 29. In unit 29 the released, gelled starch is enzymatically converted into sugars, and subsequently into ethanol. Ethanol is discharged via conduit 30 and CO₂ formed in the fermentation is discharged via conduit 31. Heat required particularly for the ethanol processing is supplied via a conduit 23 from the combined heat and power system 33.

Residual fraction from the maize plants is fed via conduit 34 to a unit 35 for the biogas fermentation. Manure is supplied via a conduit 36 and residual flows and optionally a residual fraction from the product fermentation unit 29 via conduit 37. Biogas is supplied via conduit 38 to the combined heat and power system 33. Heat required for the biogas production is supplied from the combined heat and power system 33 as indicated by arrow 39. Digestate from the biogas fermentation unit 35 is fed via a conduit 40 to a unit 41 for treatment of the digestate, and in particular for the purpose of making degradable (not yet digested) cellulose and lignocellulose which are present and which are fed back via a conduit 42 to the biogas fermentation unit 35. CO₂ and ammonia dissolved in the digestate are discharged via conduit 43. Waste water is drained via conduit 69 and dry matter (particularly sand/lignin) is discharged via conduit 70.

Electricity generated with the combined heat and power system 33 is supplied via a line 71 to the electricity grid and waste gas is discharged via conduit 72.

FIG. 3 shows a device 46 which forms a part of the device 26 according to the invention shown in the flow diagram of FIG. 2. A starch-rich fraction, maize kernels 47 are supplied in parallel to a silo 49, 50 via valves 48. In this silo 49, 50 hot water 51 supplied via valves 52 is sprayed onto the maize kernels. The hot water 51 has a temperature of about 100° C. and is heated in a heat exchange 53 to which heat is supplied from a combined heat and power system (not shown). After a sufficient gelling, the maize kernels are supplied alternately from silo 49 or 50 via a valve 54 to a release unit 55.

Water which is sprayed onto the starch kernels during gelling is discharged, after a sufficient treatment time, via valves 56 and guided via a conduit 57 through heat exchanger 53 and fed back to silos 49, 50.

In release unit 55 the gelled starch is released from its biological covering, for instance using a crusher, and transferred to unit 58 for the purpose of the enzymatic conversion of the gelled starch to sugar, and subsequently via fermentation to ethanol. It is noted at this point that between the hot water treatment and reaching unit 58 the number of micro-organisms present is drastically reduced, due to the killing action of the hot water, to a level which is substantially no longer a factor in the enzymatic starch conversion and the sugar fermentation.

Unit 58 can consist of one conversion reactor or of two or more reactors. Starch can for instance be converted in a first reactor using Maxamyl® at 90° C. and pH 7. The conversion via sugar to ethanol using amyloglucosidase and yeasts can then be realized in a subsequent unit at pH 4.5 and a temperature decreasing from 60° C. to 35° C. Heating or cooling can take place if necessary using heat exchanger 59.

The conversion medium from unit 58 is added via a conduit 62 to a separating unit 61 such as a microsieve (larger than 0.5 μm, such as 1-5 μm), centrifuge or separator/decanter. The separated starch/yeast is fed back via a conduit 62 to unit 58. The remaining liquid phase is supplied via a heat exchanger to a distillation column 65, in which 78% by volume ethanol is distilled out via heat exchanger 66 at atmospheric pressure over only a small number of distillation plates, while the supply flow contained 7.5% by volume ethanol. Via a separator 67 CO₂ is discharged via conduit 68 and ethanol via conduit 69. An underlying flow 70 obtained after passage through of one or more distillation plates is fed back via heat exchanger 53 to unit 58. Heat required for operating the distillation column 65 is fed from the combined heat and power system via heat exchanger 78. An underlying fraction of distillation column 65 is fed back via a conduit 80 and heat exchanger 63 to fermentation unit 58.

FIG. 4 shows in more detail the unit 41 for treating the digestate. The digestate is fed via conduit 40 to a separating unit 81. Waste water (or thin fraction) is discharged via conduit 69. The residual flow is split up so that a solid mass (sand/lignin) is discharged via conduit 82. The part remaining behind (or thick fraction) is fed via a conduit 71 to a heating/degassing unit 72. The degassing is performed using steam supplied via conduit 73 from the combined heat and power system 33. CO₂ and ammonia, or biogas are discharged via a conduit 76. The degassed thick fraction, which comprises cellulose/lignocellulose, is supplied via a conduit 74 to a treatment tank 75 in which calcium hydroxide is fed via conduit 76 to the hot cellulose/lignocellulose (about 85° C.). Units 72 and 75 can optionally be combined. Formed ammonia is discharged via conduit 76 and can be recovered by washing-out. Following treatment at this high temperature, the treated cellulose/lignocellulose is fed back after about one day via conduit 42 to the biogas fermentation unit 35.

Additional experiments investigated whether the gelled and released starch from the starch-rich fraction is suitable for the fermentative forming of ethanol. For this purpose a comparison is made with other carbon sources, particularly glucose and maize starch.

FIG. 5 shows that through time the CO₂ production (equivalent to ethanol formation) keeps pace with the CO₂/ethanol production on the basis of glucose and maize starch.

FIG. 6 shows the CO₂/ethanol production speed and FIG. 7 the associated glucose concentration through time after addition of the enzymes.

FIG. 8 shows in more detail the unit 41 for treating the digestate. The digestate is supplied via conduit 40 to a separating unit 81. Waste water (or thin fraction) is discharged via conduit 69. The remaining digestate (the thick fraction) is fed via conduit 71 to a heating/degassing unit 72. It is also possible to dispense with the separating unit and to feed the digestate directly to a heating/degassing unit. The degassing is performed using heat supplied from the combined heat and power system 33 via conduit 73. The heat can be supplied in the form of warm exhaust gases, but also in other forms. A part of the moisture present in the digestate can also be evaporated using the heat. The degassed digestate, which comprises cellulose/lignocellulose, is fed via conduit 74 to treatment tank 75 in which caustic solution, for instance calcium hydroxide, is fed to the hot cellulose/lignocellulose (about 85° C.) via conduit 82. Units 72 and 75 can optionally be combined. Formed ammonia, expelled CO₂ and possible exhaust gases are discharged via conduits 76 and 77. Ammonia can be recovered in the form of ammonium sulphate, ammonium nitrate or other salt by washing with a gas washer (not shown). Water can be recovered from the gases using an optional condenser 83. The recovered water can be guided via conduit 85 to the ethanol process. The rest of the gases are discharged via conduit 84.

After treatment at the high temperature the treated cellulose/lignocellulose is fed back after about a day via conduit 42 to the biogas fermentation unit 35.

### EXAMPLE 1

### Lime Treatment of Digestate

The dry digestate fraction comprises large quantities of indigestible cellulose and hemicellulose (lignocellulose). The idea is that treatment with Ca(OH)₂ should make these components available for methanogenesis. After treatment the digestate can be fed back to the digestate unit.

Sludge was taken from a digestate unit fed with maize. Part of the sludge was treated with Ca(OH)₂ until a pH of more than 11 was obtained (75-100 g Ca(OH)₂/kg DM). The forming of biogas was measured during treatment with and without Ca(OH)₂. During the treatment of pulp, the pulp was first heated to 85° C., after which lime was added. Fresh pulp without maize kernels was measured by way of comparison.

Fresh pulp (without kernels) produced 280 m³CH₄/tonne OM. Standard maize pulp will usually produce 400 m³CH₄/tonne OM.

The sludge from the maize-fed reactor produced gases during heating. During addition of the lime ammonia was formed (discernible due to the odour). About 75 to 100 g Ca(OH)₂/kg DM) was necessary to obtain a sufficiently high pH. This explains the decrease in the ratio of organic to dry matter in the samples after treatment (see table 1).

After lime treatment 240 m³CH₄/tonne OM was produced; an additional production of ((240−140=) 100 m³CH₄/tonne OM compared to the untreated digestate. It was possible for the methane yield of maize pulp without maize kernels to increase by 180 (50 m³CH₄/tonne OM) due to the lime treatment. It is calculated that the measured methane production from pulp without maize kernels (280 m³CH₄/tonne OM) implies that a degradation of 73% of all hydrocarbons occurs in the pulp (27% of the non-degraded hydrocarbons).

The increase of 18% means that 67% of the non-degradable hydrocarbons became degradable due to the lime treatment.

TABLE 1
Results of anaerobic digestion experiments before
and after lime treatment (LT)

| | Unit | | | | | |
|---|---|---|---|---|---|---|
| | | | | | | m³CH₄/ |
| | | | | m³/ton | | tonne |
| | % | % | % | gas | % | OM |
| Label | DM | OM | OM/DM | yield | CH₄ | CH₄ yield |
| Digester | 10% | 8% | 81% | 15 | 0.76 | 140 |
| Post digester | 9% | 7% | 81% | 11 | 0.76 | 120 |
| Storage | 9% | 7% | 76% | 14 | 0.74 | 150 |
| Digester after | 14% | 9% | 67% | 35 | 0.64 | 240 |
| LT | | | | | | |
| Post digester | 12% | 8% | 67% | 24 | 0.67 | 190 |
| after LT | | | | | | |
| Storage after LT | 12% | 8% | 66% | 24 | 0.72 | 220 |

The gas bubbles which escaped from the sediment during heating will be CO₂. This has an advantage on a large scale: the more CO₂ escapes during heating, the less Ca(OH)₂ is required to increase the pH. The forming of ammonia already began at low pH values as a result of the increased temperature. The more NH₄⁺ there is present in the pulp, the more lime will be required to reach high pH values; at high temperatures NH₄⁺ escapes as NH₃, whereby Ca(OH)₂ is neutralized. Because carbonate and ammonia are in the same aqueous phase, it can be advantageous, depending on the substrate, to remove as much water as possible prior to heating and the lime treatment. If the DM content were increased by 10% to 30%, the lime requirement could be decreased considerably. It is found that the lime treatment can assist in increasing the methane yield.

The above results show that the gelled and released starch from the starch-rich fraction can be converted in adequate manner by the enzymes used into sugar and subsequently into ethanol.

Although mention is only made in the exemplary embodiments of the forming of ethanol as conversion product, it will be apparent that, by using other enzymes and micro-organisms and/or applying other conditions, other desired conversion products or combinations thereof can be produced with the method and device according to the invention.

1. Method for producing electricity and conversion products such as ethanol, comprising the steps of:
i) separating a starch source into a starch-rich fraction and a residual fraction;
ii) heating the starch-rich fraction for the purpose of gelling the starch;
iii) releasing the gelled starch from the starch-rich fraction;
iva) converting the gelled starch enzymatically into sugars;
ivb) converting the sugars fermentatively into the conversion products;
v) further processing the conversion products from the conversion medium;
vi) generating biogas from residual fraction;
vii) generating electricity and heat from biogas and/or residual fraction via cogeneration of heat and electricity; and
viii) using the generated heat in one or more steps i) to vi).

2. Method as claimed in claim 1, wherein the starch-rich fraction comprises starch-rich plant organs.

3. Method as claimed in claim 1 or 2, wherein the starch-rich fraction is stored, optionally after a preservative treatment.

4. Method as claimed in claims 1-3, wherein steps ii), iii) and iv) are performed substantially under aseptic conditions.

5. Method as claimed in claims 1-4, wherein the gelled starch is pressed out of the starch-rich fraction.

6. Method as claimed in claim 5, wherein the enzymatic conversion comprises an enzymatic conversion of starch into sugars and a fermentative conversion of sugars into conversion products such as ethanol.

7. Method as claimed in claims 1-6, wherein an enzyme system is used which comprises at least one amylase and at least one amyloglucosidase.

8. Method as claimed in claims 1-7, wherein enzymes for the enzymatic conversion and/or yeasts and/or fermenting agents for the fermentative conversion are recovered.

9. Method as claimed in claims 1-8, wherein step v) comprises an ethanol distillation.

10. Method as claimed in claim 9, wherein ethanol is further processed in step v) by means of gas stripping and ethanol condensation.

11. Method as claimed in claim 10, wherein the gas stripping comprises of CO₂ stripping.

12. Method as claimed in claims 1-11, wherein CO₂ originates from step iv).

13. Method as claimed in claims 1-12, wherein the heating of the starch-rich fraction in step ii) takes place with heat generated in the combined heat and power system.

14. Method as claimed in claims 1-13, wherein the residual fraction is stored.

15. Method as claimed in claim 14, wherein the residual fraction and/or thick fraction from the biogas fermentation is treated, for instance using calcium hydroxide.

16. Method as claimed in claims 1-15, wherein the thick fraction is heated in order to remove dissolved carbon dioxide.

17. Method as claimed in claims 1-16, wherein the starch source comprises maize.

18. Method as claimed in claims 1-17, wherein formed ammonia is captured, for instance with formed carbon dioxide.

19. Device for performing the method as claimed in claims 1-18, comprising:
a unit for separating a starch source into a starch-rich fraction and a residual fraction;
a unit for heating the starch-rich fraction for the purpose of gelling the starch;
a unit for releasing gelled starch from the starch-rich fraction;
at least one unit for enzymatic conversion of gelled starch into sugars and for the fermentative conversion of sugars into conversion products;
a unit for producing biogas from residual fraction;
an associated combined heat and power unit for combined production of electrical or mechanical energy and heat from the residual fraction and/or biogas;
a unit for processing the conversion products; and
means for supplying heat from the combined heat and power unit to at least one of the other units of the device.

20. Device as claimed in claim 19, comprising a unit for storing the starch-rich fraction and/or residual fraction.

21. Device as claimed in claim 19 or 20, wherein the unit for enzymatic conversion comprises a first tank for the enzymatic conversion of starch and a second fermentation tank for the conversion to sugars and ethanol.

22. Device as claimed in claims 19-21, wherein the unit for processing conversion products comprises an ethanol distillation.

23. Device as claimed in claims 19-22, wherein the unit for processing ethanol comprises a gas stripper and an ethanol condensation unit.

24. Device as claimed in claims 19-23, comprising a unit for treating the residual fraction and/or a thick fraction from the fermentative conversion.

25. Method for producing biogas from a thick fraction of a digestate, comprising of heating the thick fraction of a digestate, allowing CO₂ to escape from the thick fraction, adding an alkaline agent to the heated thick fraction, treating the heated thick fraction with the alkaline agent and introducing the treated thick fraction into a digester for further fermentation of the treated thick fraction.

26. Method as claimed in claim 25, wherein the heating of the thick fraction takes place at a temperature between 70° C. and 100° C., preferably at a temperature between 80° C. and 90° C., more preferably at 85° C.

27. Method as claimed in claim 25 or 26, wherein the treatment of the thick fraction with the alkaline agent takes place for 6 to 48 hours, preferably for 6 to 24 hours, most preferably for 6 to 12 hours.

28. Method as claimed in any of the claims 25-27, wherein the alkaline agent is chosen from the group consisting of Ca(OH)₂, KOH, NaOH or Ca(O).

29. Method as claimed in any of the claims 25-28, wherein the pH during treatment of the thick fraction is between pH 9 and 12, preferably between pH 9.5 and pH 10.5.

30. Method as claimed in any of the claims 25-29, wherein the alkaline agent is added in a concentration of between 25 and 300 g, preferably between 25 and 150 g, most preferably between 25 and 100 g of alkaline agent per kilogram of dry matter of the thick fraction.

31. Method as claimed in any of the claims 25-30, wherein nitrogen is removed from the thick fraction by means of a gas washer.
